(19) United States
(12) Patent Application Publication
(10) Pub. No.: US 2002/0039993 A1
(43) Pub. Date: Apr. 4, 2002

(54) Use of inhibitors of the activation of CXCR4 receptor by SDF-1 in treating rheumatoid arthritis

(76) Inventors: Robert J. Winchester, New York, NY (US); Tetsunori Seki, Roosevelt Island, NY (US); Percio Saltz Gulko, Riverdale, NY (US)

Correspondence Address: Cooper & Dunham LLP, 1185 Avenue of the Americas, New York, NY, 10036, US

(21) Appl. No.: 09/773,876

(22) Filed: Jan. 31, 2001

Related U.S. Application Data
(63) Continuation of application No. PCT/US99/17178, filed on Jul. 29, 1999.
Application No. PCT/US99/17178 is a continuation-in-part of application No. 09/127,651, filed on Jul. 31, 1998, now abandoned.

(51) Int. Cl. A61K 38/17; A61K 39/395

(52) U.S. Cl. 514/2; 424/142.1; 424/133.1

(57) ABSTRACT

This invention provides a method for treating rheumatoid arthritis or other forms of inflammatory arthritis which comprises administering to a subject an amount of an agent effective to inhibit the activation of the CXCR4 receptor by SDF-1. This invention further provides a composition for treating rheumatoid arthritis comprising an effective amount of an agent capable of inhibiting the activation of the CXCR4 receptor by SDF-1 and a pharmaceutically acceptable carrier. This invention also provides a method for determining whether an agent is capable of inhibiting the activation of the CXCR4 receptor by SDF-1 comprising: (a) contacting cells expressing the CXCR4 receptor in the presence of SDF-1 with the agent under condition permitting activation of the CXCR4 receptor by SDF-1 if the agent is absent; and (b) determining whether the amount of activation of the CXCR4 receptor by SDF-1 is decreased in the presence of the agent relative to the amount of activation in its absence, such a decrease indicating that the agent is capable of inhibiting the activation of the CXCR4 receptor by SDF-1. Finally, this invention provides agents identified by such a method.

This application claims priority of U.S. Ser. No. 09/127,651, filed Jul. 31, 1998, the content of which is hereby incorporated by reference. Throughout this application various references are referred to by arabic numbers within parenthesis. Disclosures of these publications in their entireties are hereby incorporated by reference into this application to more fully describe the state of the art to which this invention pertains.

### BACKGROUND OF THE INVENTION

The architecture, cellular composition and state of cellular activation of the synovial membrane in rheumatoid arthritis have been well described(1,2), but fundamental questions still remain unanswered regarding the precise molecular nature and biologic significance of these inflammatory changes. The intimal synovial lining layer that is extensively altered in synovitis synovium through hyperplasia and infiltration is formed by the interaction of two distinct cell types: intimal synoviocytes derived from the fibroblastoid lineage and intercalated, hemopoietically-derived, monocytoid lineage cells(3-5). During histogenesis of the normal joint the lining cell apparently provides both guidance clues and receptor interactions to the specialized synovial monocytoid cells that result in its incorporation into the lining layer(1) . Together, the cells comprising the intimal layer carry out a number of functions responsible for the integrity and sustenance of the joint.

The form and function of the intimal synoviocyte apparently distinguishes them from fibroblastoid cells found deeper in the synovium, although relatively little is known about the differences between these members of the fibroblastoid lineage(6). Several genes have been identified that are selectively expressed in the normal intimal, but not subintimal synoviocytes including vascular cell adhesion molecule 1 (VCAM-1)(7), uridine diphosphoglucose dehydrogenase (UDPGD) and decay accelerating factor (DAF)(6). In chronic synovitis immunopathologic studies have shown that the fibroblastoid intimal synoviocytes respond to the events by proliferating and altering their pattern of gene expression to include expression of a variety of molecules that range from MHC class II structures, through cytokines to enzymes that directly participate in the destructive remodelling of joint tissues (1,8-13). In parallel, some of the fibroblasts in subintimal locations similarly express MHC class II and VCAM-1(6,13). However, the performance of more analytic studies of synoviocyte cell biology has been constrained because there is no basement membrane that delimits intimal synoviocytes from the subintimal fibroblastoid cells in either normal or inflamed joint tissues, and the purification and separate culture of these two potentially distinct lineages has been difficult, if not impossible.

For many years it has been recognized that long term cultures of fibroblastoid cells obtained from synovial tissue of individuals with rheumatoid arthritis and marked degrees of intimal hyperplasia continue to exhibit several phenotypes that together are characterized by varying degrees of striking ‘stellate’ or ‘dendritic’ morphology, enhanced growth, increased glucose consumption, altered adherence behavior, constitutive overproduction of metalloproteinases and the elaboration of proinflammatory cytokines(13-16). The distinctive but not entirely uniform phenotype of rheumatoid synoviocytes is not found in similarly cultured synoviocytes obtained from osteoarthritis synovia that lack lining cell hyperplasia and any inflammatory cell infiltration(16). The occurrence of this distinctive phenotype has been shown to be characteristic of, but not unique to, cell lines established from rheumatoid arthritis synovia, as it is also demonstrable in cultures initiated from a number of different entities characterized by chronic inflammation, including osteoarthritis synovia with considerable degrees of inflammation(16). These cell lines have been used to gain a series of interesting insights into the biology of joint inflammation (15-22), although the origin of the cells in culture is somewhat uncertain and at least at the time of initiation includes hyperplastic intimal synoviocytes, subintimal synoviocytes, other fibroblastoid cells as well as non mesenchymal cells that do not survive after three passages. We and others have postulated that the distinctive changes in synoviocyte phenotype observed in these cell lines mirror certain similar events occurring in the inflamed synovium itself(14,15,23-25).

Finding additional genes that may be selectively expressed in the cultured synoviocyte obtained from inflammatory synovitis would likely provide further insight into the origin of the synoviocytes comprising the cultures, the biology of the intimal synoviocyte and the alterations that this cell and other synovial fibroblasts undergo in synovitis. To further this gene discovery process, a general approach was adopted based on the construction of representational difference libraries(26,27) that had been used to clone the differences between two complex genomes. It involves a cloning procedure with PCR amplification of cDNA to generate simplified representations of the expressed genes followed by a modified subtractive step and subsequent screening to facilitate the gene identification.

By identification of these genes, it is discovered that SDF-1 is expressed on the synoviocytes which can activate the CXCP4 receptors on lymphocytes and monocytes, either causing them to enter the joint and initiate inflammation through a chemokine effect, or activate these cells that have entered the joint to enhance inflammation.

### SUMMARY OF THE INVENTION

This invention provides a method for treating rheumatoid arthritis or other forms of inflammatory arthritis which conprises administering to a subject an amount of an agent effective to inhibit the activation of the CXCR4 receptor by SDF-1.

This invention further provides a composition for treating rheumatoid arthritis comprising an effective amount of an agent capable of inhibiting the activation of the CXCR4 receptor by SDF-1 and a pharmaceutically acceptable carrier.

This invention also provides a method for determining whether an agent is capable of inhibiting the activation of a CXCR4 receptor by SDF-1 comprising: (a)contacting cells expressing the CXCR4 receptor in the presence of SDF-1 with the agent under conditions permitting activation of the CX-R4 receptor by SDF-1 if the agent is absent; and (b) determining whether activation of the CXCR4 receptor by SDF-1 is decreased in the presence of the agent relative to the amount of activation in its absence, such a decrease in the amount of activation indicating that the agent is capable of inhibiting the activation of the CXCR4 receptor by SDF-1. Finally, this invention provides agents identified by the such a method.

### BRIEF DESCRIPTION OF THE FIGURES

FIG. 1. Schematic chart describing the procedure for the identification of genes overexpressed in rheumatoid arthritis synoviocytes

FIG. 2. Comparison of the amino acid sequence of human semaphorin III, IV, V, and mouse semaphorin E with the predicted sequence of human semaphorin VI. Nucleotide sequence of the cDNA fragment of human semaphorin VI was translated into an amino acid sequence, and compared to that of the corresponding region of human semaphorin III, IV, V and mouse semaphorin E. Conserved amino acids are indicated with boxes. One amino acid gap introduced in the human semaphorin III and V to obtain the best alignment was marked by X.

FIG. 3. Comparison of amino acid sequence of the human N-acetylglucosamine-6-sulfatase and predicted amino acid sequence from the C. elegans cosmid K09C4 and the clone ts99. Nucleotide sequence of the cDNA fragment of the clone ts99 was translated to an amino acid sequence, and the corresponding region of the human N-acetylglucosamine-6-sulfatase and C. elegans cosmid K09C4 were compared. Conserved amino acids are marked with boxes.

FIG. 4. Representative Northern blot analysis of the isolated clones. lug polyA⁺ RNA was used to run on a 1% agarose gel. The probes used are clone ML2122, clone ML2115, lumican, IGFBP5, SDF-1-α, semaphorin VI, collagenase type IV. The first lane of each blot is RNA from cultured rheumatoid arthritis synoviocytes, and the second lane is RNA from cultured osteoarthritis synoviocytes.

### DETAILED DESCRIPTION OF THE INVENTION

Throughout this application, reference to specific nucleotides are to nucleotides present on the coding strand of the nucleic acid. The following standard abbreviations are used throughout the specification to indicate specific nucleotides:

| | |
|---|---|
| C = cytosine | A = adenosine |
| T = thymidine | G = guanosine |

This invention provides a method for treating rheumatoid arthritis or other forms of inflammatory arthritis which comprises administering to a subject suffering from such a condition an amount of an agent effective to inhibit the activation of a CXCR4 receptor by SDF-1, particulary the human CXCR4 receptor. Diseases which represent other forms of inflammatory arthritis are known in the art, and include, but are not limited to, psoriatic arthritis and inflammatory osteoarthritis.

In one embodiment of the invention, the agent is an oligopeptide or a polypeptide. In a further embodiment, the agent is an antibody or a portion of an antibody such as a FAB fragment. In this embodiment the antibody is preferably human, partially human, chimeric, or a humanized antibody.

In another embodiment, the agent is a nonpeptidyl agent. For example, the nonpeptidyl agent AMD3100 (Donzella, G. A., et al (1998), AMD3100, a small molecule inhibitor of HIV-1 entry via the CXCR4 co-receptor, Nature Medicine, 4:72-77).

AMD3100 is a bicyclam derivative and is representative of this class of chemicals. See DeVreese, K. et al., Antiviral Research 29, 209-219 (1996).

This invention provides a composition for treating rheumatoid arthritis comprising effective amounts of an agent capable of blocking the activation of the CXCR4 by SDF-1 and a pharmaceutically acceptable carrier. In an embodiment, the agent is oligopeptide. In another embodimet, the agent is a polypeptide. In a further embodiment, the agent is an antibody or a portion of an antibody, such as a FAB receptor. Preferably, the antibody is a human, humanized or chimeric antibody.

Pharmaceutically acceptable carriers are well-known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

The agent may be administered orally, parenterally or intra-articularly.

In another embodiment of the invention, the agent is a nonpeptidyl agent, such as the nonpeptidyl agent AMD3100.

This invention also provides a method for determining whether an agent is capable of inhibiting the activation of a CXCR4 receptor by SDF-1 comprising: (a) contacting cells expressing the CXCR4 receptor in the presence of SDF-1 with the agent under conditions permitting activation of the CXCR4 receptor by SDF-1 if the agent is absent; and (b) determining whether the amount of activation of the CXCR4 receptor by SDF-1 is decreased in the presence of the agent relative to the amount of activation in its absence, such a decrease in the amount of activation indicating that the agent is capable of inhibiting the activation of the CXCR4 receptor by SDF-1. In one embodiment, the CXCR4 receptor is a human CXCR4 receptor. In a further embodiment the cells are lymphocytes or monocytes. In yet another embodiment the CXCR4 receptor is expressed in prokaryotic or eukaryotic cells, including but not limited to bacterial, fungal, plant or animal cells using methods well known in the art.

Finally, this invention provides an agent identified by the above-described method and a composition comprising an amount of an agent identified by the above-described method effective to inhibit the activation of the CXCR4 receptor by SDF-1 and a suitable carrier.

This invention will be better understood from the Experimental Details which follow. However, one skilled in the art will readily appreciate that the specific methods and results discussed are merely illustrative of the invention as described more fully in the claims which follow thereafter.

Experimental Details

First Series of Experiments

Synoviocyte Culture.

Synovial tissue was obtained at the time of joint replacement from a classic rheumatoid arthritis with 10-12 layers of hyperplastic lining cells which intensively expressed HLA-DR and HLA-DQ molecules, and showed replacement of the superficial lining layer with monocytoid cells and an extensive subintimal infiltration of lymphocyte aggregates and monocytes. The osteoarthritis sample was taken from a synovium that had no lining cell hyperplasia and no subintimal cellular infiltration. The tissue was minced, enzymatically dissociated and cultured through five passages in Isocove's Modified Dulbeccols Media (Gibco, Grand Island, N.Y.) supplemented with selected lots of 10% fetal calf serum (Gibco, Grand Island, N.Y.) and 1% penicillin-streptomycin (Sigma, St. Louis, Mo.) as described(5). The resulting cells which presumably included intimal and subintimal synoviocytes in varying proportions according to their proportion in the starting material were grown to confluence and passaged by brief exposure to dilutions of 1% trypsin-EDTA (Sigma, St. Louis, Mo.).

Construction of the Subtraction Library and Preliminary Sequencing.

PolyA+ RNA was isolated from the fifth passage synoviocytes using a mRNA Isolation Kit (Stratagene). 2 ug of twice purified polyA+ RNA was used as a template for cDNA synthesis in the RiboClone cDNA Synthesis System (Promega). The synthesized cDNA was ligated with the oligonucleotides GATCCGCGGCCGC and GCGGCCGCGT as described(26). After selection of fragments larger than 250 nucleotides by fractionation through a Sephacryl S-400 column (Pharmacia) and phosphorylation with T4 polynucleotide kinase, the cDNA was digested with the restriction enzyme MboI. The fragments were then ligated with oligonucleotides J-Bam-24 ACCGACGTCGACTATCCATGAACG and J-Bam-12 GATCCGTTCATG, and amplified as described(26). The PCR products, after fractionation through Sephacryl S-400 column, were digested with MboI and they comprised the primary amplicon. DNA from rheumatoid arthritis synoviocytes was further ligated with oligonucleotides N-Bam-24 AGGCAACTGTGCTATCCGAGGGAG and N-Bam-12 GATCCTCCCTCG. The hybridization was performed as described(26) except that the ratio of tester and driver was kept 1:100 throughout. 10 ug of the osteoarthritis primary amplicon were hybridized with 0.1 ug of the rheumatoid arthritis primary amplicon in 5 ul of 24 mM EPPS, pH 8.0, 1 mM EDTA, 1M NaCl for 20 hr at 67 C. The hybridized DNA was subjected to 10 cycles of PCR with N-Bam-24 as a primer, followed by digestion with mung bean nuclease. One four hundredth of the digests was further amplified for 20 cycles. After digestion with MboI, the DNA product was ligated with oligonucleotides R-Bam-24 AGCACTCTCCAGCCTCTCACCGAG and R-Bam-12 GATCCTCGGTGA. Hybridization and amplification steps were repeated. After redigestion with MboI, the resulting differentially subtracted cDNA fragments were cloned into a BamHI site of the plasmid pUC18. The recombinants were inoculated in an ordered grid pattern on nitrocellulose filters, which were then probed with the osteoarthritis cDNA amplicon ³²P-labeled with the Megaprime DNA labeling System (Amersham). The DNA sequence of the non-hybridized recombinants was determined in an Applied Biosystems DNA Sequencer Model 373A or 377 using standard dye terminator chemistry. The seqman module of the Lasergene program (DNAstar) was used for identification of homologous recombinants and grouping them into groups. The Genman module of the Lasergene program was used to search the GenBank databases including the expressed sequence tag database on CDROM. BLAST was used to verify the identification of sequences that showed no homology with entries in the CDROM database.

Northern Blot Analysis.

Probes were prepared from the clones by PCR amplification of the inserts, digestion with MboI and isolation by electrophoresis on a 1% agarose gel. 1ug of the once purified polyA+ RNA of the same preparation used for the construction of subtraction library was run on a 1% agarose gel, containing 1.9% formaldehyde and hybridized with the ³²P-labelled probes as described(28). The membranes were re-probed several times after stripping off the previous probe.

Construction of a Rheumatoid Arthritis cDNA Library.

The same preparation of the cDNA from the rheumatoid arthritis patient used for the construction of the subtraction library was ligated with EcoRI adapters. These constructs were cloned into λgt10 by standard procedures and the library was screened as described previously(29).

Experimental Results

Identification of Genes Differently Represented in the Cultured Rheumatoid Arthritis and Osteoarthritis Synoviocytes.

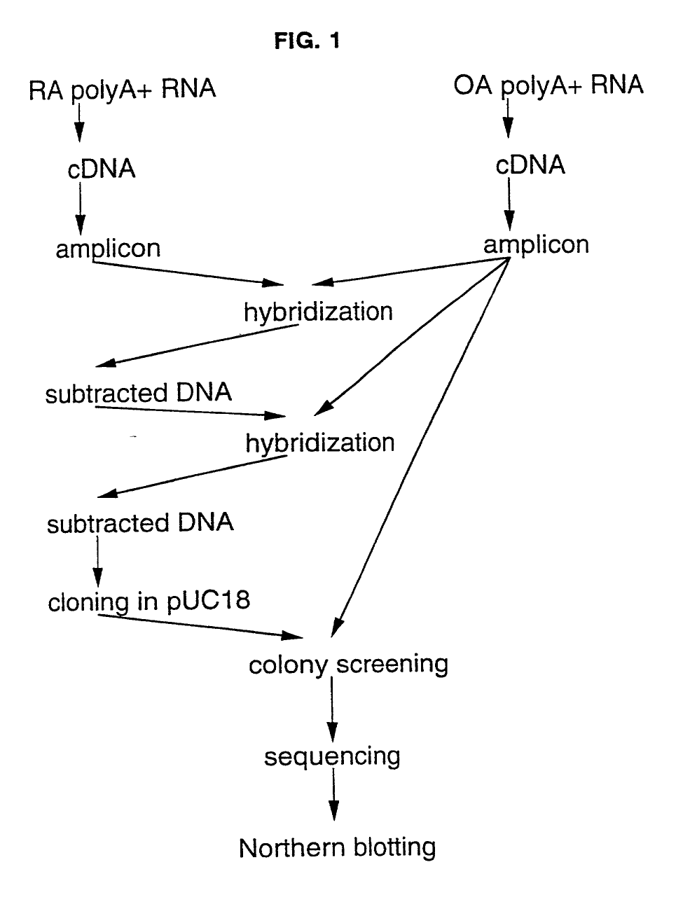

To identify genes that may be differently expressed in the cultured rheumatoid arthritis and osteoarthritis synoviocytes, cell lines originating from a carefully selected highly inflammatory rheumatoid arthritis synovium and an osteoarthritis synovium with no lining cell hyperplasia or inflammatory cell infiltration were chosen. Two subtraction cycles were performed between polyA+ RNA from fifth passage rheumatoid arthritis and osteoarthritis synoviocytes followed by negative screening of the resulting difference representation clones with a probe consisting of the ³²P-labelled osteoarthritis synovial fibroblastoid cDNA amplicons (FIG. 1). 319 recombinant clones were selected for further analysis by DNA sequencing.

Nucleotide sequencing revealed that many of those 319 recombinants had the same sequence, comprising of distinct 24 sequence groups. As would be expected, the number of recombinants representing each group varied considerably, ranging from just one to as many as 77 recombinants (Table 1). Comparison of the sequence with the GenBank database revealed that 16 sequence groups showed more than 97% homology with the previously identified human genes (Table 1). In the case of insulin-like growth factor binding protein 5 (IGFBP5) and interferon-inducible 56 kd protein (II56kd protein) two CDNA fragments derived from the different portion of the same gene.

TABLE 1
List of the identified genes and number of
obtained clones.

| Name of gene | Number of Clones |
|---|---|
| Group 1 * | |
| Manganese superoxide dismutase | 8 |
| Collagenase type IV | 4 |
| Complement factor B | 4 |
| α-B crystallin | 1 |
| Interferon-gamma IEF SSP 5111 | 1 |
| B94 protein | |
| HLA-E heavy chain | 1 |
| NMB protein | 9 |
| Muscle fatty-acid-binding protein | 1 |
| Group 2 * | |
| VCAM-1 | 2 |
| II56 kd protein | 42 |
| 71 kd 2′-5′-oligoadenylate | 1 |
| synthetase | |
| Mac2 binding protein | 21 |
| Biglycan | 16 |
| Lumican | 3 |
| IGFBP5 | 107 |
| SDF-1-α | 69 |
| Semaphorin VI | 1 |

On Northern blot analysis, Group 1 genes showed little difference in the intensity of hybridization between cultured rheumatoid and osteoarthritis synoviocyte RNA. Group 2 genes exhibited overexpression in rheumatoid arthritis synoviocytes compared with osteoarthritis synoviocyte. In the case of the genes that were represented in two different sequence groups, a total number of clones are shown in the table. Those genes are II56 kd protein, 8+34, IGFBP5, 30+77, and SDF-1α 28+41.

Characterization of Novel Genes.

Of the remaining 8 sequence groups, two highly represented clones with copy numbers of 28 and 41 in the library had 32% and 25% similarity, respectively, to the 3′-untranslated region of the mouse SDF-1α. These fragments hybridized with the same clones from the λgt10 rheumatoid arthritis synoviocyte library, indicating that they derived from the same transcript.

The nucleotide sequence of the clones showed high homology with mouse SDF-1α in the coding region (data not shown), and was almost identical with the subsequently published sequence of the human SDF-1α gene(30).

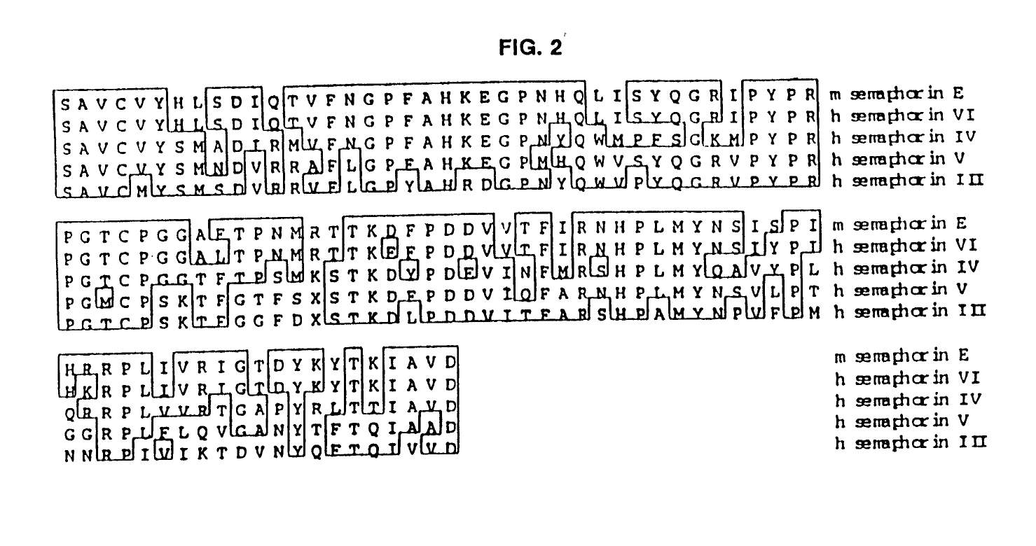

Another clone was found to have 90% homology with mouse semaphorin E at the nucleotide level and 94% at the putative amino acid level. This suggested that the isolated clone was a human homologue of the mouse semaphorin E, and it was tentatively named human “semaphorin VI”. A comparison of the amino acid sequences with the previously described human semaphorins III, IV, V and mouse semaphorin E is shown in FIG. 2.

Analysis of another clone showed some homology at the nucleotide level and more significantly at the putative amino acid sequence level with a variety of sulfatases.

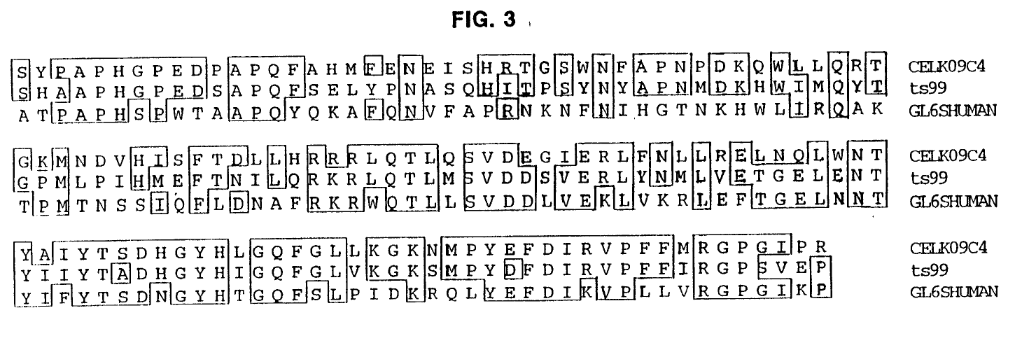

Among human genes the greatest similarity was with the human N-acetyl-glycosamine sulfatase. However the sequence of this clone was most homologous with the putative amino acid sequence derived from the C. elegans genomic cosmid K09C (FIG. 3).

A portion of the sequence of clone ML2115 was 99% identical with the EST sequence AA447232. The remaining clones did not show significant homology to any known genes in either nucleotide level nor in translated amino acid level, and their identification is continuing.

Northern Analysis.

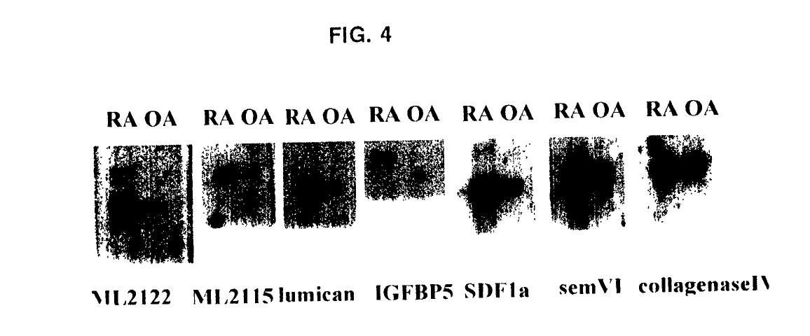

To determine the actual difference in level of expression of the genes characterized by the 24 different recombinant clones, Northern analysis of polyA+ RNA from the two cell lines used to make the difference library was performed. The level of GAPDH expression was not detectably different between both synoviocytes (data not shown). FIG. 4 illustrates a representative gel using inserts of clones as probes from, lumican, IGFBP5, SDF-1α, semaphorin VI, collagenase type IV and the two clones, ML2122 and ML2115 which did not show appreciative homology to the known genes. As shown, the expression of collagenase type IV did not differ significantly between the two RNA preparation. Similarly, the expression of genes depicted in Group 1, Table 1, such as HLA-E, α-B-crystallin and manganese superoxide dismutase had minimally increased or essentially equivalent levels of expression in the osteoarthritis and rheumatoid arthritis synoviocyte cell lines.

However, of the genes identified in this study, 11 had moderate to marked differentially elevated expression in the rheumatoid arthritis synoviocyte line used for the subtraction (Table 1. Group 2), suggesting that these genes were constitutively overexpressed in cultured rheumatoid arthritis synoviocytes. These 11 genes included: VCAM-1, Mac-2 binding protein (Mac-2BP), IGFBP5, biglycan, lumican, SDF-1α, II56 kd protein, 71 kd 2′-5′ oligoadenylate synthetase, semaphorin VI, and two clones ML2115 and ML2122. The clone ML2115 hybridized with a 6 kb mRNA. The clone ML2122 hybridized with three species of mRNA of which 4.7 kb was the major one (FIG. 4). The characterization of these clones is being continued.

Since SDF-1α has an alternatively spliced form SDF-1-βwith which it shares the most of coding region but a different 3′-untranslated region(30), the expression of SDF-1β was investigated. Its expression was also found to be increased in parallel with that of SDF-1α in the rheumatoid arthritis synoviocytes compared to the osteoarthritis cells (data not shown)

Experimental Discussion

The objective of the present study was to develop a method to identify additional genes that comprise the distinctive biochemical and cell physiologic phenotype of cultured rheumatoid arthritis fibroblastoid synoviocytes. Of 24 genes characterized by this procedure, 11 were found to be constitutively overexpressed by Northern analysis in the rheumatoid arthritis synoviocyte culture used for subtraction and three were novel genes. The relatively unbiased gene discovery approach used to subtract differential representations of the expressed genes in the two prototype cell lines is a general method useful for the identification of differentially expressed genes. The characteristics of the genes identified in the present study direct increased attention to the possibilities that synoviocytes from synovia with marked lining cell hyperplasia are characterized both by different matrix and cell-cell interactions and by the fact that they likely provide guidance clues and sites for receptor interaction to infiltrating monocytes and lymphocytes during normal histogenesis of the synovial lining, providing a mechanism for the location of monocyte lineage cells in the intimal layer. Moreover, in an exaggerated mode of leukocyte ingress that could occur during synovial hyperplasia, these gene products might foster the localization of an immune or autoimmune response to the joint. Taken together the results direct further attention to the role of mesenchymal cells in immune-mediated diseases.

In the present experiments special attention was directed to the selection of the tissue source of the two cell lines used in the subtraction. Prior studies showed that cell lines obtained from patients clinically characterized as osteoarthritis with various degrees of inflammatory synovitis elaborated proinflammatory cytokines in patterns often similar to those found in rheumatoid arthritis samples(16,25). In this study the reference synovial sample was from a patient with osteoarthritis who had no evidence of synovitis with only a single cell layer of intimal synoviocytes. In contrast the rheumatoid arthritis synovium used for gene isolation had 10-12 layers of hyperplastic lining cells. It should be stressed that a limitation of this study is that it is not possible to identify the site of origin in the synovial lining of the cultured synoviocytes, although application of reagents directed to identification of these products of these genes in situ should facilitate resolving the question of their origin.

The gene discovery approach used in this work was initially developed to detect the absolute difference between two genomes where each gene is present in the same ratio (26). Because of the differences in the number of each mRNA species and the likelihood that the frequencies of certain mRNA species relatively differed between cultured rheumatoid arthritis and osteoarthritis synoviocytes, the subtraction steps were modified by reducing the ratio of the tester and driver DNA. This had the effect of decreasing the completeness of the subtraction step, but increasing the possibility of discovering genes expressed at a variety of different levels in the two cell lines. To compensate for any potential inefficiency of subtraction, a negative selection screening step was added using the driver osteoarthritis synoviocyte cDNA amplicon as a probe, and the constitutive increase in expression of the identified genes was confirmed in Northern analysis.

Several technical points require comment. The cDNA synthesis was primed with oligo (dT) to bias the ultimate library towards one rich in 3′-untranslated regions, because the nucleotide sequence of this region is more divergent than that of the coding regions. The restriction enzyme MboI was chosen to create DNA fragments of relatively small size to facilitate efficient and even amplification by PCR, and to increases the chance of isolating genes which are differentially spliced and/or members of a supergene family. The DNA fragments were fractionated through a Sephacryl S-400 column to avoid biased amplification of numerous fragments smaller than 250 nucleotides.

The subtractive method is less influenced by differences in a low copy number mRNA species than the related differential display method, however the number of recombinants analyzed places a sampling error limit on the identification of a rare species. In the present study, some differentially expressed genes were identified only by the presence of a single recombinant. There are additional technical reasons, such as the absence of appropriate Mbo 1 sites why some genes previously expressed in cultured inflammatory synoviocytes might not be identified(16,25,31).

Of the 11 genes constitutively increased in expression in the rheumatoid synoviocytes, VCAM-1, a 110 kd member of the immunoglobulin gene superfamily, and Mac-2BP, also termed ‘90k tumor associated protein’, both exhibit properties that suggest they could mediate heterotypic binding of monocyte-lineage intimal synoviocytes to fibroblastoid lineage synoviocytes. VCAM-1 has been previously described as markedly increased on rheumatoid arthritis synoviocytes(1,23) and the identification of VCAM-1 by this difference method supports the validity of this gene discovery approach for intimal synoviocytes. VCAM-1 binds circulating monocytes and lymphocytes expressing the α₄β₁ (VLA4) integrin. Mac-2BP, a heavily N-glycosylated secreted protein which binds stoichiometrically to the macrophage-associated lectin Mac-2 (galectin-3)(32,33), also binds to the monocyte CD14 structure in the presence of LPS and LBP(34). Binding of Mac-2BP to these receptors initiates monocyte-lineage cells to secrete IL-1 and IL-6 and increases their expression of ICAM-1(35,36). This alteration in monocyte state could be one of the factors modulating the cell into a synovial lining macrophage.

The overexpression of the semaphorin VI by synoviocytes is intriguing because the semaphorins are a family of transmembrane signalling and secreted guidance glycoprotein molecules that are implicated in directing axonal extension (37). However, in view of the relatively small number of axons in the synovium, it seems unlikely that the physiologic role of the semaphorin VI molecule is to signal through an axonal receptor. Rather, one might conjecture semaphorin VI plays some role in chemotaxis of monocytes and their differentiation. Suggesting a broader role of semaphorin molecules in cellular interaction, CD100 which plays a role in B-cell activation parallel to that of CD40 ligand has recently been identified as a member of this family(38). A report of the overexpression of semaphorin VI gene in rheumatoid arthritis synovial fibroblastoid cells by the differential display method appeared while this manuscript was in preparation(39).

Another molecule constitutively expressed by the rheumatoid synoviocyte was the chemokine SDF-1α. It was first identified as a pre-B cell growth stimulating factor produced by marrow stromal cells (40,41). SDF-1-α attracts pro- and pre-B cells(42) as well as CD34+hematopoietic progenitor cells(43). Mice genetically deficient for SDF-1α lack B-cells and have hematopoiesis only in their liver(44). SDF-1α is the ligand for the CXCR-4 chemokine receptor that serves as a co receptor for entry of T-tropic syncytial inducing forms of HIV into T-cells(45). SDF-1α has recently been the subject of an interesting series of studies that demonstrated this chemokine to be a highly efficacious transendothelial chemoattractant for both monocytes and T-lymphocytes(46). It is not clear that SDF-1β has a biologic activity different from that of SDF-1α at the moment. We speculate that the production of SDF-1 by intimal synoviocytes in the normal joint could act as a guidance cue for the continual entrance into the intimal synovial membrane of monocyte lineage precursors committed to differentiation into phagocytic lining cells. Similarly SDF-1 and other chemokines elaborated by the normal synoviocytes may act to enhance the ingress of lymphocytes into the joint tissues to facilitate physiologic surveillance functions.

Several genes were identified as constitutively expressed, indicating the possibility of altered cell-matrix interactions as part of the distinctive rheumatoid arthritis synoviocyte phenotype. Lumican is a keratan sulfate proteoglycan that plays a critical role in the basis of corneal transparency(47). In adult cartilage lumican exists predominantly in a glycoprotein form lacking keratan sulfate (48). Macrophages do not adhere to intact corneal keratan sulfate proteoglycans but attach and spread rapidly on the lumican core protein after the removal of keratan sulfate chains(49). This observation suggests some species of lumican could also act to localize macrophages to sites of the synovium. Biglycan, a dermatan sulfate-proteoglycan, is both induced by TGF-β, and binds TGF-β (50), suggesting that biglycan may down regulate TGF-β activity by sequestering this growth factor in the extracellular matrix. IL-6 stimulates the expression of biglycan, while TNF-α depresses its expression(51). IGFBP5, was the most highly represented species in the difference library. This molecule increases IGF-1 binding to the fibroblast membrane by attaching to the extracellular-matrix proteins, types III and IV collagen, laminin and fibronectin(52). IGFBP5 may have an antiinflammatory role that opposes the effect exhibited by IL-1 and TNF-α of stimulating proteoglycan degradation and decreasing proteoglycan synthesis (53). The observation that IGFBP5 is further induced by exposure of cells to prostaglandin E2(54) is of interest with respect to the pattern of morphologic change and gene activation observed in synoviocyte cultures upon addition of this agent(55).

The 71 kd 2′-5′ oligoadenylate synthetase is a subunit of one of several interferon-induced enzymes that, when activated by double-stranded RNA, convert ATP into 2′-5′ linked oligomers of adenosine(56). The interferon-inducible 56 kd protein is of unknown function, but in common with 2′-5′ oligoadenylate synthetase is strongly induced by interferons(57). The expression of these two genes directs attention to the presence of activation-like features in the phenotype of the rheumatoid arthritis synoviocytes.

In prior studies it was found that the relative overexpression of known genes comprising the distinctive phenotype of cultured inflammatory synoviocytes varied somewhat from cell line to cell line (16,25). Preliminary evidence using these newly isolated genes indicates similar sample to sample variation in the relative degree of expression of one overexpressed gene relative to another by Northern analysis. Similarly, additional studies will be required to determine whether the levels of expression of the remaining genes that were not preferentially overexpressed in rheumatoid synoviocytes distinguish synoviocytes in general from fibroblastoid cells in other anatomic sites.

The identification of a group of constitutively overexpressed genes in this study is relevant to the three principal cell biologic possibilities explaining the origin of the distinctive phenotype of these cultured rheumatoid synoviocytes. We and others have postulated that the phenotype could result from sustained modulation of gene expression in several fibroblast lineage cells of the joint that developed as a response to prolonged paracrine signalling through products of a local immune response, analogous to a phenotypic imprinting process(2). A second possibility is that the cells are primarily ‘transformed’ as suggested by Gay and colleagues(9). However, perhaps most likely in view of the features of the genes isolated in this study, is a third possibility that the phenotype exhibited by these cells is similar to that of the normal intimal synoviocyte. Thus at the start of an experiment, a culture derived from rheumatoid arthritis synovia characterized by marked intimal synoviocyte hyperplasia would contain an increased proportion of intimal lining synoviocytes that are responsible for the resulting phenotype of the cultured cells because of their lineage difference in patterns of gene expression.

Each of these three potential origins shares in common the possibility that the presence of increased quantities of these guidance and cell interaction molecules may itself create a novel synovial microenvironment that could facilitate interactions with monocyte lineage cells and foster the entry of large numbers of inflammatory and immune leukocytes. The first two mechanisms imply that the contribution of synoviocytes to the cell biologic basis of synovitis is qualitatively based due to the presence of abnormally activated or modulated cells while the third mechanism implies a quantitative over representation of members of a normal cell lineage that physiologically exhibits distinctive properties. In each case, the resulting environment may modulate or deviate an ongoing immune response and reenforce its subsequent evolution into an autoimmune process.

Since inflammatory imprinting or hyperplasia could be initiated by a non specific minor traumatic event or even driven by a local immune response to a common pathogen, this might provide a non antigen-specific mechanism for localizing potential pathogenic immune responses to the joint. For example, an additional action of SDF-1 at higher concentrations could be the facilitation of earlier stages of peripheral B-cell development in the synovial milieu that are relevant to the presence and maturation of abundant B-cells in the rheumatoid synovium and to their production of rheumatoid factors(58) Furthermore, several additional molecules produced by the synoviocyte can interact to facilitate other aspects of B-cell development. IL-6, a cytokine with effects on B-cell differentiation, is constitutively increased in synoviocytes obtained from rheumatoid arthritis patients(16) and its synthesis by monocytes is induced by Mac-2BP, as described above. Interleukin 7-dependent proliferation of pre-B cells is also enhanced upon exposure to biglycan(59). Similarly these molecules could attract and facilitate interaction with and activation of monocytes. For example, Mac-2BP which induces homotypic monocyte aggregation and activation(33) could be a factor present in supernatants from cultured rheumatoid arthritis synoviocytes that induces blood monocytes to form giant cells (60). Thus, along with the variety of genes that mediate the well recognized effector functions of matrix remodelling and tissue destruction(55), the genes expressed by the mesenchymal cells of the joint may affect antigen non specific immune localization or amplification mechanisms that could play a role in the puzzling phenomenon of why localized joint inflammation develops in many disparate diseases in the setting of immune responses that apparently have little to do with the joint.

### REFERENECES

2. Winchester R Rheumatoid Arthritis. Edited by M M Frank, K F Austen, H N Claman, E R Unanue. Sampter's Immunological Diseases: Boston, Mass.: Little, Brown and Company; 1995.

3. Barland P, Novikoff A B, Hamerman D Electron microscopy of the human synovial membrane. J Cell Biol 1962;14:207-220.

4. Norton W L, Ziff M Electron microscopic observations in the rheumatoid synovial membrane. Arthritis Rheum 1966;9:589-610.

5. Burmester G R, Dimitriu-Bona A, Waters S J, Winchester R J Identification of three major synovial lining cell populations by monoclonal antibodies directed to Ia antigens and antigens associated with monocytes/macrophages and fibroblasts. Scand J Immunol 1983;17:69-82.

6. Edwards J C, Leigh R D, Cambridge G Expression of molecules involved in B lymphocyte survival and differentiation by synovial fibroblasts. Clin Exp Immunol 1997;108:407-414.

7. Morales-Ducret J, Wayner E, Elices M J, Alvaro-Gracia J M, Zvaifler N J, Firestein G S Alpha 4/beta 1 integrin (VLA4) ligands in arthritis. Vascular cell adhesion molecule-1 expression in synovium and on fibroblast-like synoviocytes. J Immunol 1992;149:1424-1431.

8. Klareskog L, Forsum U, Scheynius A, Kabelitz D, Wigzell H Evidence in support of a self-perpetuating HLA-DR-dependent delayed-type cell reaction in rheumatoid arthritis. Proc Natl Acad Sci USA 1982;79:3632-3636.

9. Trabandt A, Aicher W K, Gay R E, Sukhatme V P, Nilson Hamilton M, Hamilton R T, et al Expression of the collagenolytic and Ras-induced cysteine proteinase cathepsin L and proliferation-associated oncogenes in synovial cells of MRL/I mice and patients with rheumatoid arthritis. Matrix 1990;10:349-361.

10. Firestein G S, Zvaifler N J How important are T cells in chronic rheumatoid synovitis? Arthritis Rheum 1990;33:768-73.

11. Arend W P, Dayer J M Cytokines and cytokine inhibitors or antagonists in rheumatoid arthritis. Arthritis Rheum 1990;33:305-15.

12. Koch A E, Kunkel S L, Burrows J C, Evanoff H L, Haines G K, Pope R M, et al Synovial tissue macrophage as a source of the chemotactic cytokine IL-8. J Immunol 1991;147:2187-2195.

13. Winchester R J, Burmester G R Demonstration of Ia antigens on certain dendritic cells and on a novel elongate cell found in human synovial tissue. Scand J Immunol 1981;14:439-444.

14. Werb Z, Mainardi C L, Vater C A, Harris E D, Jr. Endogenous activation of latent collagenase by rheumatoid synovial cells. N Engl J Med 1977;296:1017.

15. Castor C W, Ritchie J C, Scott M E, Whitney S L Connective tissue activation X1: stimulation of glycosaminoglycan and DNA formation by a platelet factor. Arthritis Rheum 1977;20:859-868.

16. Bucala R, Ritchlin C, Winchester R, Cerami A Constitutive production of inflammatory and mitogenic cytokines by rheumatoid synovial fibroblasts. J Exp Med 1991;173(3):569-574.

17. Smith C A Properties of synovial cells In culture. J Exp Med 1971;134:306S.

18. Wynne-Roberts C R, Castor C W Ultrastructural comparison of rheumatoid and nonrheumatoid synovial fibroblasts grown in tissue culture. Arthritis Rheum 1972;15:65-83.

19. Anastassiades T P, Len J, Wood A, Irwin D The growth kinetics of synovial fibroblastic cells from inflammatory and noninflammatory arthropathies. Arthritis Rheum 1978;21:461-466.

20. Ponteziere C, Desmoulins D, Agneray J, Ekindjian O G, Cals M J Comparative proliferation of non-rheumatoid and rheumatoid human synovial cells. Int J Tissue React 1990;12:229-236.

21. Goddard D H, Grossman S L, Moore M E Autocrine regulation of rheumatoid arthritis synovial cell growth in vitro. Cytokine 1990;2:149-155.

22. Winchester R, Su F, Ritchlin C Modulation of synoviocytes by inflammation—source of a persistent non-immunologic drive in synovitis: analysis of levels of mRNA expression by a simple multi-gene assay. Clin Exp Rheumatol 1993;11(S8):87-90.

23. Kriegsmann J, Keyszer G M, Geiler T, Brauer R, Gay R E, Gay S Expression of vascular cell adhesion molecule-1 mRNA and protein in rheumatoid synovium demonstrated by in situ hybridization and immunohistochemistry. Lab Invest 1995;72:209-214.

24. Ritchlin C T, Winchester R J Potential mechanisms for coordinate gene activation in the rheumatoid synoviocyte: implications and hypotheses. Springer Semin Immunopathol 1989;11:219-234.

25. Ritchlin C, Dwyer E, Bucala R, Winchester R Sustained and distinctive patterens of gene activation in synovial fibroblasts and whole synovial tissue obtained from inflammatory synovitis. Scand J Immunol 1994;40:292-8.

26. Lisitsyn N, Wigler M Cloning the differences between two complex genomes. Science 1993;259:946-951.

27. Hubank M, Schatz D G Identifying differences in mRNA expression by representational difference analysis of cDNA. Nucleic Acids Res 1994;22:5640-5648.

28. Sambrook J, Fritsch E F, Maniatis T: Molecular Cloning: A laboratory Manual. 2nd edition. Cold Spring Harbor, N.Y., Cold Spring Harbor Laboratory, 1989

29. Seki T Identification of multiple isoforms of the low-affinity human IgG Fc receptor. Immunogenetics 1989;30:5-12.

30. Shirozu M, Nakano T, Inazawa J, Tashiro K, Tada H, Shinohara T, et al Structure and chromosomal localization of the human stromal cell- derived factor 1 (SDF1) gene. Genomics 1995;28:495-500.

31. Tan PL, Farmiloe S, Yeoman S, Watson J D Expression of the interleukin 6 gene in rheumatoid synovial fibroblasts. J Rheumatol 1990;17:1608-1612.

32. Koths K, Taylor E, Halenbeck R, Casipit C, Wang A Cloning and characterization of a human Mac-2-binding protein, a new member of the superfamily defined by the macrophage scavenger receptor cysteine-rich domain. J Biol Chem 1993;268:14245-14249.

33. Inohara H, Akahani S, Koths K, Raz A Interaction between galectin-3 and Mac-2-binding protein mediate cell-cell adhesion. Cancer Res 1996;56:4530-4534.

34. Yu B, Wright SD LPS-dependent interaction of Mac-2-blnding protein with immobilized CD14. J Inflamm 1995;45:115-125.

35. Iacobelli S, Arno E, D'Orazio A, Coletti G Detection of antigen recognized by a novel monoclonal antibody in tissue and serum from patients with breast cancer.

Cancer Res 1986;46:3005-3010.

36. Ullrich A, Sures I, D'Egidio M, Jallal B, Powell T J, Herbst R, et al The secreted tumor-associated antigen 90K is a potent immune stimulator. J Biol Chem 1994;269:18401-18407.

37. Luo Y, Shepherd I, Li J, Renzi M J, Chang S, Raper J A A family of molecules related to collapsin in the embryonic chick nervous system. Neuron 1995;14:1131-1140.

38. Hall K T, Boumsell L, Schultze J L, Boussiotis V A, Dorfman D M, Cardoso A A, et al Human CD100, a novel leukocyte semaphorin that promotes B-cell aggregation and differentiation. Proc Natl Acad Sci USA 1996;93:11780-5.

39. Mangasser-Stephan K, Dooley S, Welter C, Mutschler W, Hanselmann RG Identification of human semaphorin E gene expression on rheumatoid synovial cells by mRNA differential display. Biochem Biophys Res Commun 1997;234:153-156.

40. Nagasawa T, Kikutani H, Kishimoto T Molecular cloning and structure of a pre-B-cell growth-stimulating factor. Proc Natl Acad Sci USA 1994;91:2305-2309.

41. Tashiro K, Tada H, Heilker R, Shirozu M, Nakano T, Honjo T Signal sequence trap: a cloning strategy for secreted proteins and type I membrane proteins.

Science 1993;261:600-603.

42. D'Apuzzo M, Rolink A, Loetscher M, Hoxie J A, Clark-Lewis I, Melchers F, et al The chemokine SDF-1, stromal cell-derived factor 1, attracts early stage B cell precursors via the chemokine receptor CXCR4. Eur J Immunol 1997;27:1788-1793.

43. Ajuti A, Webb I J, Bleul C, Springer T, Gutierrez-Ramos J C The chemokine SDF-1 is a chemoattractant for human CD34+ hematopoietic progenitor cells and provides a new mechanism to explain the mobilization of CD34+ progenitors to peripheral blood. J Exp Med 1997;185:111-120.

44. Nagasawa T, Hirota S, Tachibana K, Takakura N, Nishikawa S, Kitamura Y, et al Defects of B-cell lymphopoiesis and bone-marrow myelopoiesis in mice lacking the CXC chemokine PBSF/SDF-1. Nature 1996;382:635-8.

45. Bleul C C, Farzan M, Choe H, Parolin C, Clark-Lewis I, Sodroski J, et al The lymphocyte chemoattractant SDF-1 is a ligand for LESTR/fusin and blocks HIV-1 entry. Nature 1996;382:829-32.

46. Bleul C C, Fuhlbrigge R C, Casasnovas J M, Aiuti A, Springer T A A highly efficacious lymphocyte chemoattractant, stromal cell-derived factor 1 (SDF-1). J Exp Med 1996;184:1101-9.

47. Rada J A, Cornuet P K, Hassell J R Regulation of corneal collagen fibrillogenesis in vitro by corneal keratan sulfate proteoglycan (lumican) and decorin core proteins. Exp Eye Res 1993;56:635-48.

48. Grover J, Chen Z N, Korenberg J R, Roughley P J The human lumican gene. Organization, chromosomal location, and expression in articular cartilage. J Biol Chem 1995;270:21942-21949.

49. Funderburgh J L, Mitchler R R, Funderburgh M L, Roth M R, Chapes S K, Conrad G W Macrophage receptors for lumican. Invest Ophthalmol Vis Sci 1997;38:1159-1167.

50. Hildebrand A, Romaris M, Rasmussen L M, Heinegard D, Twardzik D R, Border W A, et al Interaction of the small interstitial proteoglycans biglycan, decorin and fibromodulin with transforming growth factor beta. Biochem J 1994;302:527-534.

51. Ungefroren H, Krull N B Transcriptional regulation of the human biglycan gene. J Biol Chem 1996;271:15787-15795.

52. Jones J I, Gockerman A, Busby W H, Jr, Camacho-Hubner C, Clemmons D R Extracellular matrix contains insulin-like growth factor binding protein-5: potentiation of the effects of IGF-I. J Cell Biol 1993;121:679-687.

53. Tyler J A Insulin-like growth factor 1 can decrease degradation and promote synthesis of proteoglycan in cartilage exposed to cytokines. Biochem J 1989;260:543-548.

54. Pash J M, Canalis E Transcriptional regulation of insulin-like growth factor-binding protein-5 by prostaglandin E2 in osteoblast cells. Endocrinology 1996;137:2375-2382.

55. Krane S M, Dayer J M, Simon L S, Byrne M S Mononuclear cell-conditioned medium containing mononuclear cell factor (MCF), homologous with interleukin 1, stimulates collagen and fibronectin synthesis by adherent rheumatoid synovial cells: effects of prostaglandin E2 and indomethacin. Coll-Relat-Res 1985;5:99-117.

56. Marie I, Hovanessian A G The 69-kDa 2-5A synthetase is composed of two homologous and adjacent functional domains. J Biol Chem 1992;267:9933-9939.

57. Wathelet M, Moutschen S, Defilippi P, Cravador A, Collet M, Huez G, et al Molecular cloning, full-length sequence and preliminary characterization of a 56-kDa protein induced by human interferons. Eur J Biochem 1986;155:11-17.

58. Mellors R C, et. al. Rheumatoid factor and the pathogenesis of rheumatoid arthritis. J Exp Med 1961;113:475.

59. Oritani K, Kincade P W Identification of stromal cell products that intereact with pre-B cells. J Cell Biol 1996;134:771-782.

60. Grimley P M, Sokoloff L Synovial giant cells in rheumatoid arthritis. Am J Pathol 1966;49:931.

Second Series of Experiments

Immunolocalization of SDF-1 and CXCR-4 to Different Cells in the Joints of Patients with Rheumatoid Arthritis.

Objective:

In support of the prior observation of the synthesis of SDF-1 on Northern analysis by cultured synovial lining cells from rheumatoid arthritis and other forms of inflammatory arthritis, the synovial tissues of patients with rheumatoid arthritis were studies using a polyclonal goat anti SDF-1 antibody. Similarly, the tissue was studied for the expression of CXCR4, the receptor for SDF-1.

Results:

The hyperplastic layer of fibroblastoid synovial lining cells showed intense staining for the presence of SDF-1. The lymphocytes and monocytes infiltrating in the sub lining cell region of the joint exhibited intense staining for the expression of CXCR4. Similarly, the monocyte-lineage cell in the synovial lining, but not the fibrolastoid synovial lining cells also expressed CXCR4.

Interpretation:

The observations are consistence with the first series of experiments. That SDF-1 is made by fibroblastoid synovial lining cells and that this chemokine attracts lymphocytes and monocytes into the joint tissue to cause join inflammation.

Third Series of Experiments

Expression of Chemokine SDF-1 by Intimal Synoviocytes

The chemokine stromal derived factor-1 (SDF-1) was first identified as a pre-B cell growth stimulating factor produced by marrow stromal cells necessary for its population by pro- and pre-B cells and CD34+ hematopoietic progenitor cells. SDF-1 has known to be a highly efficacious transendothelial chemooattractant for monocytes and T-cells. The SDF-1 receptor, CxCR4, also serves as a co-receptor for HIV entry into T cells. We identified SDF-1 as a gene overexpressed by cultured synovial fibroblastoid cells from an individual with rheumatoid arthritis (RA) compared with those from osteoarthritis (OA) by differential subtraction. To investigate whether SDF-1 is generally overexpressed in RA synovial fibroblastoid cell lines, Northern analysis was performed with RNA from fibroblastoid cell lines of 11 RA and 2 OA samples. 8 of the RA lines were from synova with marked lining cell hyperplasia, massive inflammatory infiltration and neovasculization. All 8 exhibited moderate to marked overexpression of SDF-1. The remaining 3 RA individuals had only mild infiltration with little lining cell hyperplasia but considerable neovasculization. These 3 RA and 2 noninflammatory OA cell lines had much lower expression of SDF-1, suggesting a correlation between the level of SDF-1 expression in synoviocyte lines and features of the tissue from which they were derived. Staining of synovial tissues from 3 OA and 2 RA synovia with a polyclonal antibody to SDF-1 revealed 60-70% positivity of intimal synoviocytes in OA. In RA there was markedly stronger and more extensive SDF-1 staining in the hyperplastic lining with additional staining of some subintimal fibroblastoid cells. The results suggest that increased SDF-1 elaboration by intimal synoviocytes and possible other fibroblastoid cells may participate in the pathology of RA by enhancing recruitment of monocytes and T-lymphocytes into the synovium.

What is claimed is:

1. A method for treating rheumatoid arthritis or other forms of inflammatory arthritis which comprises administering to a subject an amount of an agent effective to inhibit the activation of the CXCR4 receptor by SDF-1.

2. The method of claim 1, wherein the agent is oligopeptide or a polypeptide.

3. The method of claim 1, wherein the agent is an antibody or a portion of an antibody.

4. The method of claim 3, wherein the antibody is a human, chimeric or humanized antibody or portion thereof.

5. The method of claim 1, wherein the agent is a nonpeptidyl agent.

6. The method of claim 6, wherein the nonpeptidyl agent is a bicyclam such as AMD3100.

7. A composition for treating rheumatoid arthritis comprising an effective amount of an agent capable of inhibiting the activation of the CXCR4 receptor by SDF-1 and a pharmaceutically acceptable carrier.

8. The composition of claim 7, wherein the agent is oligopeptide or a polypeptide.

9. The composition of claim 7, wherein the agent is an antibody or a portion of an antibody.

10. The composition of claim 9, wherein the antibody is a human chimeric, or humanized antibody.

11. The composition of claim 7, wherein the agent is a nonpeptidyl agent.

12. The composition of claim 11, wherein the nonpeptidyl agent is a bicyclam such as AMD3100.

13. A method for determining whether an agent is capable of inhibiting the activation of a CXCR4 receptor by SDF-1 comprising:
(a) contacting the cells expressing CXCR4 receptor in the presence of SDF-1 with the agent under condition permitting activation of the CXCR4 by SDF-1 if the agent is absent; and
(b) determining whether the amount of activation of the CXCR4 receptor by SDF-1 is decreased in the presence of the agent relative to the amount of activators in its absence, such a decrease in the amount of activation indicating that the agent is capable of inhibiting the activation of the CXCR4 receptor by SDF-1.

14. The method of claim 13, wherein the cells are lymphocytes or monocytes.

15. The method of claim 13, wherein the cells are bacterial, fungal, plant, or animal cells.

16. An agent identified by the method of claim 13.

17. A composition comprising an amount of an agent identified by the method of claim 13 effective to inhibit the activation of the CXCR4 receptor by SDF-1 and a suitable carrier.
